(19) United States
(12) Patent Application Publication
(10) Pub. No.: US 2015/0073516 A1
(43) Pub. Date: Mar. 12, 2015

(54) Evaporative Therapeutic Hypothermia Device

(71) Applicant: THE JOHNS HOPKINS UNIVERSITY, Baltimore, MD (US)

(72) Inventors: Soumyadipta Acharya, Baltimore, MD (US); Robert Allen, Baltimore, MD (US); Winston J. Aw, San Francisco, CA (US); Samrie Beshah, Baltimore, MD (US); Michael V. Johnston, Baltimore, MD (US); John J. Kim, Baltimore, MD (US); Robert Kim, Baltimore, MD (US); Ryan Wai Yan Lee, Baltimore, MD (US); Erika M. Moore, Highlands Ranch, CO (US); Neil P. O'Donnell, Baltimore, MD (US); Youseph Yazdi, Ellicott City, MD (US); Simon Ammanuel, Richardson, TX (US); Nathan Buchbinder, Marlboro, NJ (US)

(73) Assignee: THE JOHNS HOPKINS UNIVERSITY, Baltimore, MD (US)

(21) Appl. No.: 14/391,424

(22) PCT Filed: Apr. 9, 2013
(86) PCT No.: PCT/US2013/035729

§ 371 Date: Oct. 9, 2014

Related U.S. Application Data
(60) Provisional application No. 61/621,697, filed on Apr. 9, 2012.

(51) Int. Cl. A61F 7/10; A61F 7/08

(52) U.S. Cl. CPC A61F 7/106; A61F 7/08; A61F 2007/0095
USPC 607/114; 607/96

(57) ABSTRACT

The present invention provides a low-cost, low-power therapeutic hypothermia device for use in developing nations. The device includes a first and second receptacle separated by a space filled with a porous material such as sand. A cooling chemical can also be added to the porous material in order to speed cooling. Water is added to the porous material and a neonate is placed into the device for therapeutic hypothermia treatment. The neonate is monitored carefully using temperature sensors and a feedback system integrated into the device. Cooling can be modulated and/or warming commenced by adding Styrofoam blocks to raise the neonate off the surface of the device.

### CROSS REFERENCE TO RELATED APPLICATIONS

This application claims the benefit of U.S. Provisional Patent Application No. 61/621,697 filed on Apr. 9, 2012, which is incorporated by reference, herein, in its entirety.

### FIELD OF THE INVENTION

The present invention relates generally to neonatal care. More particularly the present invention relates to a device for providing therapeutic hypothermia to a neonate.

### BACKGROUND OF THE INVENTION

Hypoxic ischemic encephalopathy (HIE) is a serious condition that leads to death and disabilities in neonates due to oxygen deficiency in the brain. Asphyxia in neonates can be caused by a variety of factors such as maternal malnutrition, placental abruption, cord prolapse, and uterine rupture. On a global scale, between 50% and 89% of infants who suffer from severe HIE die, while many of the survivors are subject to cerebral or neural related disorders. Additionally, there is 20% to 37% mortality and morbidity in those diagnosed with moderate HIE. Because of the steep differences in HIE severity, HIE has become a major concern worldwide, and the high chances of poor outcome for those suffering from the disease suggest a significant need for improved treatment. This is especially true in developing countries, where the rate of being diagnosed with HIE is as high as 1.5% of newborns.

Studies have shown that the use of therapeutic hypothermia not only reduces the risk of death but also the possibility of long-term disability for infants who survive birth asphyxia. By slowing down the formation of free radicals and preventing apoptosis and necrosis in neurons, hypothermia has been proven to be a neuroprotective mechanism against HIE within 6 hours of birth. After 6 hours, however, neuroprotection is seemingly lost, which minimizes the effectiveness of the treatment and could instead result in adverse effects. In addition, the infant must be at least 35 weeks of gestation and weigh more than 1800 grams in order to be considered for therapeutic hypothermia.

Therapeutic hypothermia treatments do exist, and are the standards of care in many developed nations. In the United States, the current procedure for therapeutic hypothermia is a whole body cooling in which the infant is placed on a cooling blanket with an esophageal temperature probe inserted into the nose for a total of 72 hours. While on the blanket, the baby is cooled using a temperature between 3° C. and 5° C. Once the baby reaches a core temperature of 34° C., cooling is done in a servo manner to reach the target temperature of 33.5° C. to avoid overcooling. After the target temperature is maintained for a period of 72 hours, an 8 to 10 hour rewarming process begins during which the baby is warmed at a gradual rate of 0.5° C. per hour until it reaches a core temperature of 36.5° C. and stabilizes.

Currently, therapeutic hypothermia treatments are not a viable standard of care in developing nations. Existing treatments are too expensive and have an electricity demand that surpasses the availability of power in many countries. Attempts to develop low-cost, low-energy therapeutic hypothermia devices have been unsuccessful. Examples of such attempts include the use of fans in South Africa, which lacked any method of control, and the implementation of cold water bottles around the baby in Uganda, which resulted in overshoot and an increase in side effects (i.e. coagulopathy) and mortality.

It would therefore be advantageous to provide a device that provides low-cost, low-power therapeutic hypothermia for use in developing nations.

### SUMMARY

According to a first aspect of the present invention a device for providing therapeutic hypothermia to a neonate includes a first receptacle, having a first wall defining a first inner volume. The device also includes a second receptacle which is configured to sit within the first inner volume of the first receptacle. The second receptacle has a second wall defining a second inner volume, and the second inner volume is configured to receive the neonate. A third inner volume is defined between the first wall of the first receptacle and the second wall of the second receptacle, and a porous material is disposed in the third inner volume. A first sensor is configured to take a temperature of a skin of the neonate, and a second sensor is configured to take a rectal temperature of the neonate.

In accordance with an aspect of the present invention, the first receptacle takes the form of a clay pot. The second receptacle can also take the form of a clay pot. However, the second receptacle can also take the form of a basket formed from a natural fiber. The porous material filling the third volume is sand, and in some embodiments can include a cooling material such as ammonium nitrate. The device also includes a water reservoir. A biocompatible liner is disposed in the second receptacle to form a layer of protection between the second receptacle and the neonate.

In accordance with another aspect of the present invention, the device can include a visual display of the temperature of the neonate. More particularly, the device can include a temperature control system having a microprocessor receiving information from the first and second sensors. In such a case, a visual display of the temperature, is also included, and the visual display of the temperature is controlled by the microprocessor of the temperature control system. The visual display of the temperature further includes LED lights and/or an auditory alarm alert. An elevation system, such as a block, configured to raise the neonate off of a surface of the second receptacle is also included. Additionally, the device can include a heart rate monitor and a spO₂ monitor. The device can use battery power or generator power. A warming blanket can be included for the neonate.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIGS. 1A and 1B illustrate perspective views of a device to provide therapeutic hypothermia, according to an embodiment of the present invention.

FIG. 2 illustrates a schematic diagram of therapeutic hypothermia cooling, according to an embodiment of the present invention.

FIG. 3A illustrates a schematic diagram of a pathway of temperature change, according to an embodiment of the present invention.

FIGS. 3B and 3C illustrate schematic diagrams of the device's control systems for cooling and warming, according to an embodiment of the present invention.

FIG. 4 illustrates a schematic diagram of a control system according to an embodiment of the present invention.

FIG. 5 illustrates a schematic diagram representing a method of cooling and warming a neonate using the device of the present invention.

FIGS. 6A-6D illustrates various metrics of temperature in the exemplary embodiment.

FIGS. 7A-7C illustrate results for three piglets in the exemplary embodiment of the present invention.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention provides a low-cost, low-power therapeutic hypothermia device for use in developing nations. The device includes a first and second receptacle separated by a space filled with a porous material such as sand. A cooling chemical can also be added to the porous material in order to speed cooling. Water is added to the porous material and a neonate is placed into the device for therapeutic hypothermia treatment. The neonate is monitored carefully using temperature sensors and a feedback system integrated into the device. Cooling can be modulated and/or warming commenced by adding Styrofoam blocks to raise the neonate off the surface of the device.

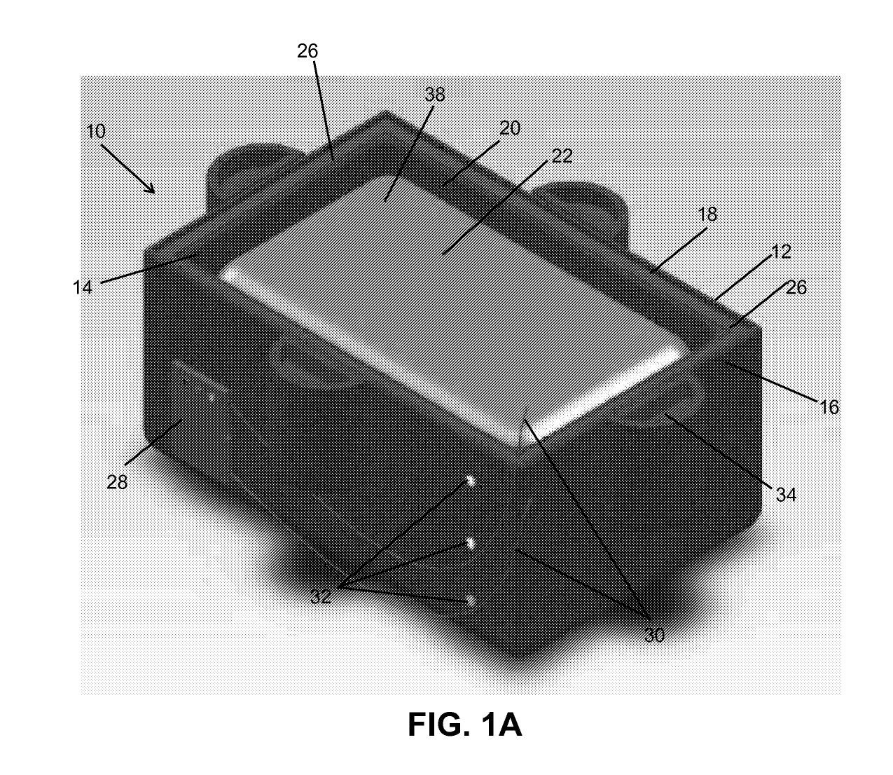

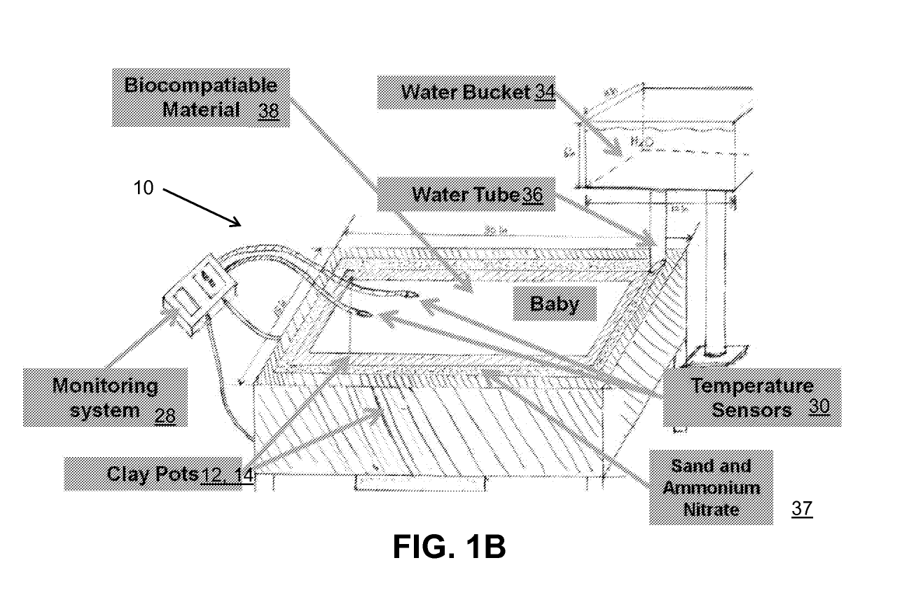

FIGS. 1A and 1B illustrate perspective views of a device to provide therapeutic hypothermia, according to an embodiment of the present invention. As illustrated in FIGS. 1A and 1B, the device 10 includes a first receptacle 12 and a second receptacle 14. The first receptacle 12 has a wall 16 defining a first inner volume 18. The second receptacle 14 is configured to sit within the first inner volume 18 of the first receptacle 12. The second receptacle 14 also has a wall 20 defining a second inner volume 22. The second inner volume 22 is configured to receive an infant for therapeutic hypothermia. The first and second receptacles can take the form of clay pots or any other suitable form known to or conceivable by one of skill in the art. Alternately, the first receptacle 12 can take the form of a clay pot and the second receptacle 14 can take the form of a basket lined with plastic or other suitable material.

As illustrated in FIGS. 1A and 1B, a space 26 is also defined between the wall 20 of the second receptacle 14 and the wall 16 of the first receptacle 12. This space 26 is filled with a porous material 26, such as sand. A urea-based powder, such as ammonium nitrate can also be added to the sand mixture to further increase heat transfer in the system. While sand and a urea-based powder are provided as examples herein, any suitable porous material and heat transfer enhancement chemical known to one of skill in the art could also be used.

The device 10 can also include a polyethylene covering for an inner surface 38 of the wall 20 of the second receptacle 14. While polyethylene is provided as an example, any biocompatible, covering material known to one of skill in the art could be used. In one exemplary embodiment the second receptacle 14 has approximate dimensions of 16 inches×12 inches×6 inches and the first receptacle 12 has approximate dimensions of 17 inches×13 inches×9 inches. The device 10 can also include a temperature monitoring system 28, having a microprocessor (not shown), thermistors or temperature sensors 30, batteries (not shown), such as two AAA batteries, circuit board (not shown), and LED lights 32. Power can alternately be provided by a generator or other electrical system. The temperature monitoring system 28 can be configured to measure rectal and skin temperature of an infant. The temperature sensors can take the form of temperature probes or skin temperature detectors, or any other sensor known to or conceivable by one of skill in the art. Other sensors can also be included in order to monitor the neonate's heart rate and spO₂.

As illustrated in FIG. 1B the device can also include a water reservoir 34 with a water tube 36 to store and convey water to the porous material between the first and second receptacles 12, 14. The water reservoir 34 and water tube 36 keep the porous material hydrated during the therapeutic hypothermia process. Alternately, the water reservoirs 34 can be integrated into the device 10, as illustrated in FIG. 1A, where multiple reservoirs 34 surround the first receptacle 12. A biocompatible lining 38 is used to protect the baby within the device 10.

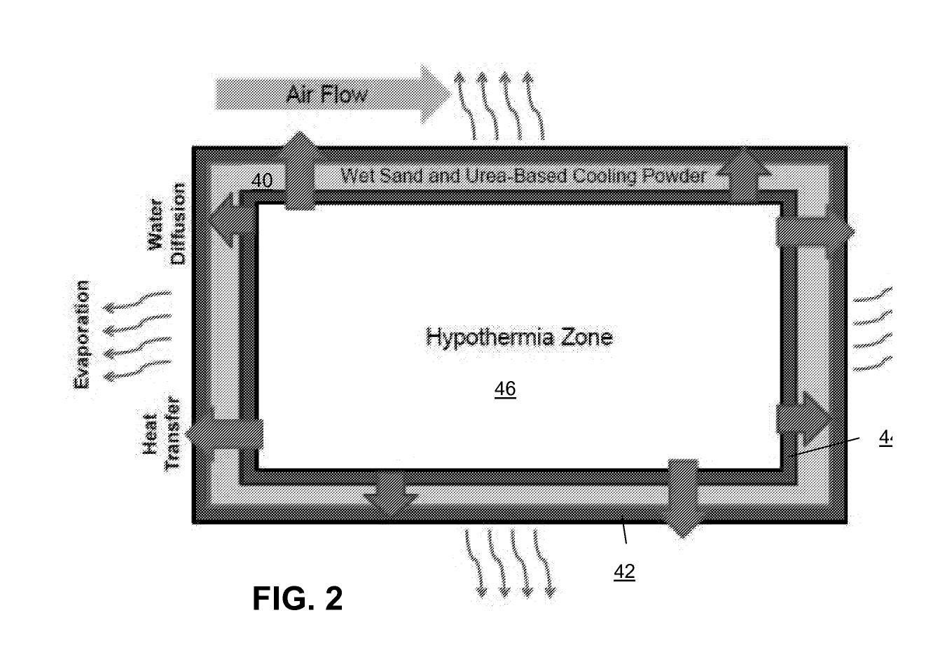

FIG. 2 illustrates a schematic diagram of therapeutic hypothermia cooling, according to an embodiment of the present invention. With regard to the present invention, the lowering of the neonate's core temperature is achieved through the use of evaporative cooling. A porous material 40 such as wet sand is placed between two receptacles 42, 44. As illustrated in FIG. 2, when water is added to the sand, the water particles slowly leave the outer receptacle 42 through small pores in the clay. As the water evaporates, heat is drawn from the inner receptacle 44, resulting in a lower temperature on a surface 46 of the inner receptacle 44.

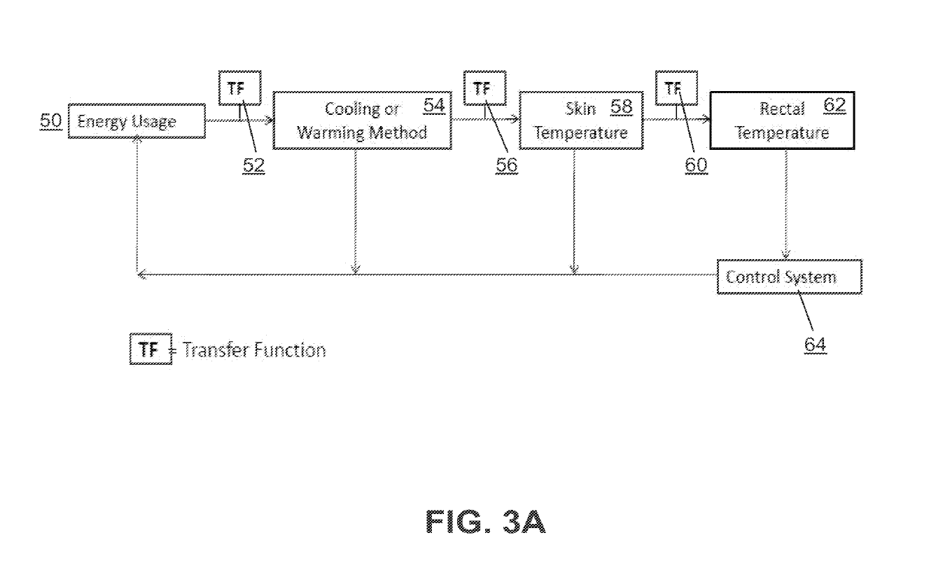

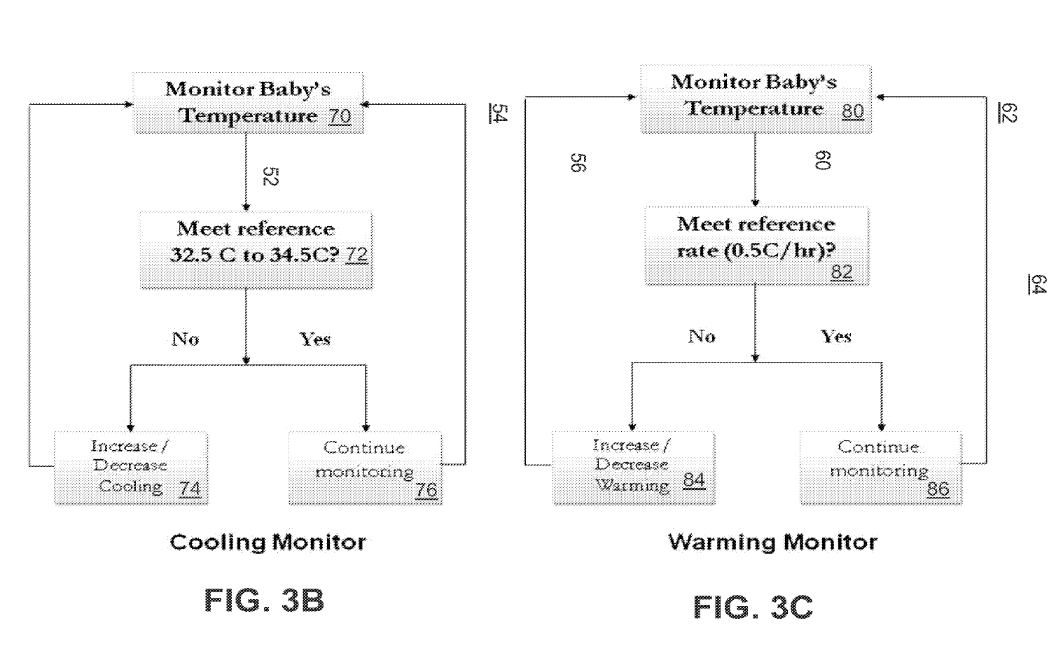

FIG. 3A illustrates a schematic diagram of a pathway of temperature change, according to an embodiment of the present invention. A step 50 includes energy usage and a first transfer function 52 converts this energy usage to cooling or warming in step 54. A second transfer function 56 converts the cooling or warming 54 to a change in skin temperature 58. A third transfer function 60 converts the change in skin temperature 58 to a change in rectal temperature 62. A control system 64 monitors this change in rectal temperature to determine how long this loop should be executed in order to reach and maintain the optimal temperature for the particular neonate. Each step is modeled with transfer functions. An inner receptacle surface temperature of 17° C. is sufficient to lower the inner body temperature of the neonate to 33.5° C. Mathematical models indicate that this decrease in the neonate's temperature takes approximately one and a half hours.

FIGS. 3B and 3C illustrate schematic diagrams of the device's control systems for cooling and warming, according to an embodiment of the present invention. FIG. 3B illustrates a step 70 of monitoring a baby's temperature and a step 72 of determining whether the temperature meets the reference temperature of 32.5-34.5° C. If no, step 74 includes increasing heating or cooling of the baby, and, if yes, step 76 includes continued monitoring of the baby. FIG. 3C illustrates a step 80 of monitoring the baby's temperature and a step 82 of determining whether the temperature meets a reference rate of 0.5° C./hr. If no, step 84 includes increasing heating or cooling of the baby, and, if yes, step 86 includes continued monitoring of the baby.

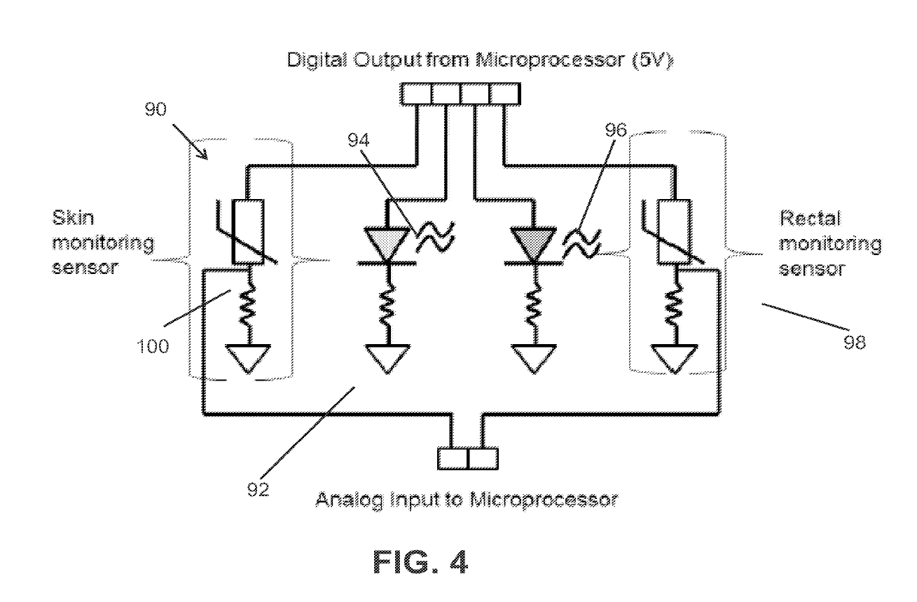

FIG. 4 illustrates a schematic diagram of a control system according to an embodiment of the present invention. The control system 90 functions primarily through a peripheral interface controllers (PIC) microcontroller from FIG. 1A and first and second LED alert lights 94, 96. Preferably, the LED alert lights 94, 96 are yellow and green in color, respectively, however, any suitable color indicator can also be used. Alerts can also take the form of sound or other means of alerting a technician that action should be taken. The microcontroller is programmed in conjunction with an individualized circuit containing thermistors 98, 100 to provide temperature feedback and to help achieve the specific temperature ranges that the neonate must be kept in during the cooling and warming processes. The first LED alert light 94 indicates that the baby's core temperature is either falling too low or rising too high, while the second LED alert light 96 indicates that the baby's core temperature is increasing at a maximum rate of 0.5° C./hr. In addition, there are three supplemental LED lights on the side of the outer pot corresponding to three different heights at which the baby may be elevated or lowered, as illustrated in FIG. 1B. For example, if the baby is cooled too fast, the top LED light will turn on to indicate that the baby needs to be elevated to the maximum height to reduce cooling. On the other hand, if the baby is being warmed at a rate faster than 0.5° C./hr, then the middle or the lowest LED light will be turned on to alert a nurse to lower the baby for effective treatment to continue. Our control system, which many developing world devices currently lack, is a simple and elegant yet requires minimal input from a health care professional.

FIG. 4 further illustrates the diagram of the control system circuit 90. The microprocessor 28 uses a temperature reading from a rectal monitoring sensor 98 to determine which LED light to turn on. A first LED alert light 94 will be turned on to warn a nurse that the rectal temperature is not within the desired temperature range, while a second LED alert light 96 will be turned on if the rectal temperature is within 33.5±1° C. during cooling. A skin monitoring sensor 100 is used as an additional safeguard to prevent any drastic change in temperature and dangers caused by a failure of rectal monitoring. The skin sensor 100 is placed on a patient's abdomen to make sure that the neonate's skin temperature is not too low or too high. The same indicating LEDs 94, 96 are used to indicate temperature variability to healthcare providers.

After the neonate has been cooled to the predetermined temperature, the neonate must then be warmed. A maximum rate of warming of 0.5° C. is required in order to avoid health risks associated with rapid rewarming Therefore, controlled passive warming is used to reduce the possibility of overshoot in warming. Passive warming allows the rate of temperature increase to occur more gradually, and also reduces the amount of energy required to operate the device.

In order to initiate warming, water is no longer added to the sand. This allows passive warming to occur more readily. Passive warming is controlled by raising and lowering the neonate out of and into the device. In order to raise the neonate's temperature, the neonate is lifted, and a small block, such as a Styrofoam block is placed underneath the baby inside the inner pot. Raising the neonate lifts it from the cool surface of the clay, allowing the neonate to undergo passive warming. Additional blocks can be added inside the inner pot to further increase the warming rate. On the other hand, to slow down the rate of temperature increase or to maintain a stable cool temperature, blocks can be removed to lower the neonate back to the inner pot. Therefore, lowering or raising the neonate from the inner pot using the blocks allows us to utilize the temperature gradient of the inner pot to regulate the neonate's core body temperature.

Passive warming might not be sufficient for warming a neonate. In such a case, an active warming process is required. A simple and cost-effective way to implement an active warming is via Kangaroo Mother Care (KMC). KMC is a World Health Organization promoted technique in which the neonate is held close to the chest of the mother, or an attending nurse to allow excessive heat to transfer from the caretaker to the neonate. Should the neonate receiving therapeutic hypothermia treatment require a large increase in temperature, KMC will be applied to the newborn.

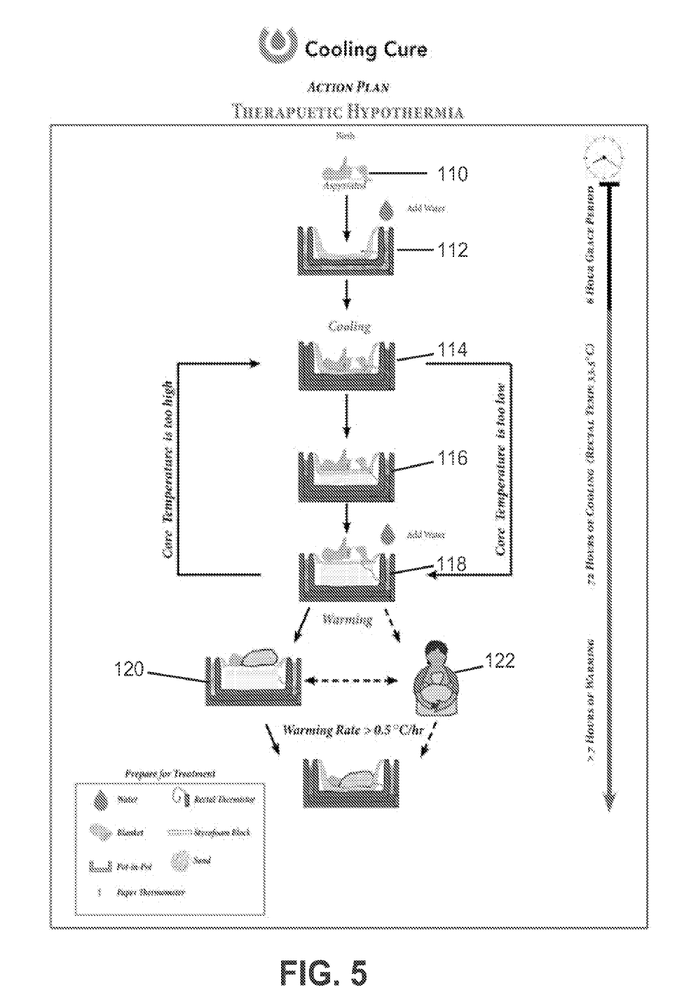

FIG. 5 illustrates a schematic diagram representing a method of cooling and warming a neonate using the device of the present invention. A neonate born asphyxiated is identified in step 110. The neonate is placed into a device according to the present invention as described with respect to FIGS. 1A and 1B in step 112 and water is added to the device. Step 114 includes cooling the neonate, while using thermometers to determine whether the neonate's temperature is too high or too low. Step 116 includes adding a Styrofoam block in order to raise the temperature of the neonate, and step 118 includes adding a second Styrofoam block in order to raise the temperature of the neonate further. After the neonate is cooled to the appropriate temperature, warming can begin. In step 120 the neonate remains on the Styrofoam blocks in the device and is wrapped in a blanket for warming In order to achieve more aggressive warming, KMC can also be used, as illustrated in step 122, such that the neonate is warmed at a rate of >0.5° C./hr. It should be noted that there is a 6 hour grace period in which to begin cooling the neonate. Cooling should extend for approximately 72 hours, during which time the neonate's temperature is kept at 33.5°, and warming should be performed for >7 hours.

### EXAMPLE

An exemplary implementation of the present invention is described herein, in order to further illustrate the present invention. The exemplary implementation is included merely as an example and is not meant to be considered limiting. Any implementation of the present invention on any suitable subject known to or conceivable by one of skill in the art could also be used, and is considered within the scope of this application.

In order to test the more practical efficacy of the present invention, three piglets (n=3; 2-10 days old; 1800±400 g), were used in a proof of concept experiment. Piglets were used as a model for neonates, because the stage of neuronal development is similar to that of a neonate. Piglets are anesthetized by breathing 5% Isoflurane in a 70/30 nitrous oxide/oxygen mixture by face mask. A tracheotomy is performed, and the lungs are mechanically ventilated with 1.5% Isoflurane in a 70/30 nitrous oxide/oxygen mixture. A rectal temperature probe is placed. Piglets undergo aseptic surgery for placement of sterile catheters into the femoral artery and vein through an incision in the groin. A solution of 5% dextrose and 0.45% saline are infused at a maintenance rate of 4 mL/kg/h. Pharmaceutical grade fentanyl is infused (20 mcg/kg+20 mcg/kg/h, IV). Pharmaceutical grade pancuronium is administered (0.2 mg/kg+0.2 mg/kg/h, IV) to facilitate electrocauterization of the muscle layers and to prevent shivering with hypothermia and rewarming The Isoflurane concentration will be increased, additional fentanyl boluses (20 mcg/kg) will be administered, and the fentanyl infusion will increased for animal comfort if the animal's heart rate exceeds 200 beats per minute (bpm) without any other apparent cause (such as hypoventilation) or if blood pressure or heart rate increase by 10% or more during surgery. (A normal heart rate for a piglet is approximately 140-200 bpm).

Throughout piglet testing, temperature is checked at least every 10 minutes. To set up the device, sand and half a urea-based cooling packet powder are mixed and placed in between the two pots. To initiate cooling, 600 mL of tap water (24-39° C.) are added to the sand and urea-based powder layer, with care taken to avoid spilling water into the inner pot. The piglet is cooled until it reaches 34° C., at which point it should be elevated fully and a blanket is placed on top for passive warming Once every half hour, a heating blanket was placed on the piglet for 10 minutes in order to mimic KMC. It was found that 10 minutes of heating allows the temperature of the piglet to increase at the correct rate. After warming for 10 minutes with the heating blanket, the piglet is placed back in the receptacle. Elevation is changed in order to maintain the new temperature achieved with the heating blanket. Even while the piglet is elevated, an inner pot temperature of 17-19° C. should be maintained. If the pot starts to warm, more water is added.

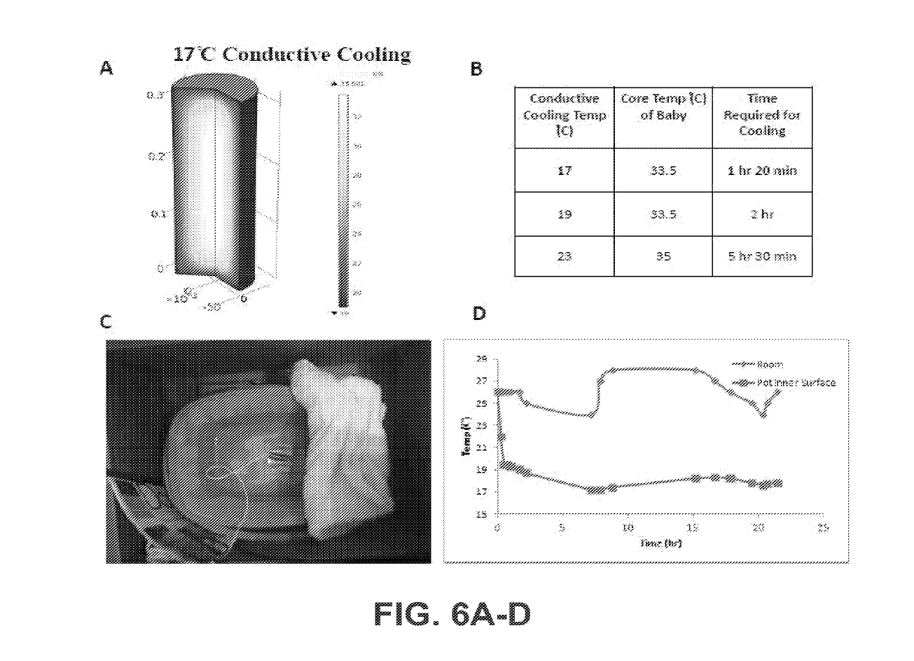

By placing wet sand between two receptacles, adding water to the sand, and measuring room and inner-pot temperatures, the effectiveness of cooling was determined. FIGS. 6A-6D illustrates various metrics of temperature in the exemplary embodiment. FIG. 6B illustrates that the inner pot surface temperature was able to reach 17° C. within 1 hour, which is the target temperature required to cool down 33.5° C. of a neonate. In addition, the device maintained this temperature without much variability, even with changes in room temperature, for over 24 hours, at which point the experiment was stopped, as illustrated in FIG. 6D.

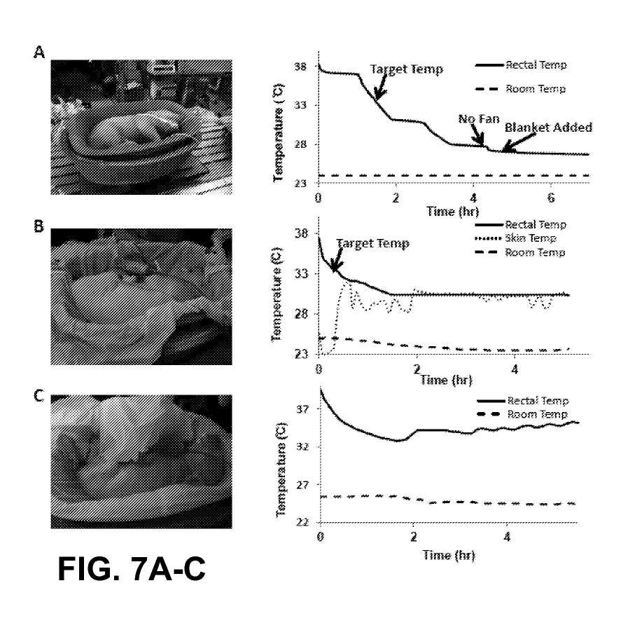

FIGS. 7A-7C illustrate results for three piglets in the exemplary embodiment of the present invention. With the first piglet, as illustrated in FIG. 7A, only the ability to cool piglets was tested. It was found that the first piglet reached the target rectal temperature in 1 hour and 45 minutes, approximately matching the mathematical modeling predictions. However, there was an overshoot in cooling, indicating that passive warming is required to prevent overcooling, as illustrated in FIG. 7A.

The second piglet tested elevation as a passive warming method. The target temperature was reached in about 25 minutes. However, the piglet was also underweight. Temperature stabilized at around 30.4° C. once passive warming had been started, and this temperature was maintained for over 3 hours, as illustrate in FIG. 7B.

During the third piglet test, passive warming was started early to prevent the overshoot seen in previous trials. The piglet reached the target temperature of 33.5° C. around 45 minutes and there was no overshoot in the cooling. Though it was not allowed to test KMC on piglet due to the animal protocol, we calculated the amount of heat flow required to simulate KMC (Supplementary FIG. 2). When the simulation was tested with a heating blanket, it was found that the core temperature is kept constant at the warmer temperature after the application of KMC for 50 minutes. Therefore, the results satisfied the warming constraint of 0.5 c/hr, as illustrated in FIG. 7C.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A device for providing therapeutic hypothermia to a neonate comprising:
a first receptacle, having a first wall defining a first inner volume;
a second receptacle, configured to sit within the first inner volume of the first receptacle, wherein the second receptacle has a second wall defining a second inner volume, wherein the second inner volume is configured to receive the neonate;
wherein a third inner volume is defined between the first wall of the first receptacle and the second wall of the second receptacle;
a porous material disposed in the third inner volume;
a first sensor configured to take a temperature of a skin of the neonate; and
a second sensor configured to take a rectal temperature of the neonate.

2. The device of claim 1 wherein the first receptacle comprises a clay pot.

3. The device of claim 1 wherein the second receptacle comprises a clay pot.

4. The device of claim 1 wherein the second receptacle comprises a basket.

5. The device of claim 4 wherein the basket comprises a natural fiber.

6. The device of claim 1 wherein the porous material comprises sand.

7. The device of claim 1 wherein the porous material comprises sand and ammonium nitrate.

8. The device of claim 1 further comprising a water reservoir.

9. The device of claim 1 further comprising a biocompatible liner disposed in the second receptacle to form a layer of protection between the second receptacle and the neonate.

10. The device of claim 1 further comprising a visual display of the temperature of the neonate.

11. The device of claim 1 further comprising a temperature control system comprising a microprocessor receiving information from the first and second sensors.

12. The device of claim 11 further comprising a visual display of the temperature, wherein the visual display of the temperature is controlled by the microprocessor of the temperature control system.

13. The device of claim 12 wherein the visual display of the temperature further comprises LED lights.

14. The device of claim 1 further comprising an auditory alarm alert.

15. The device of claim 1 further comprising an elevating device configured to raise the neonate off of a surface of the second receptacle.

16. The device of claim 1 further comprising a heart rate monitor.

17. The device of claim 1 further comprising a spO₂ monitor.

18. The device of claim 1 further comprising battery power.

19. The device of claim 1 further comprising generator power.

20. The device of claim 1 further comprising a warming blanket for the neonate.
